(19) United States
(12) Patent Application Publication
(10) Pub. No.: US 2012/0209392 A1
(43) Pub. Date: Aug. 16, 2012

(54) ADJUSTABLE REVERSE SHOULDER PROSTHESES

(75) Inventors: Laurent Angibaud, Gainesville, FL (US); Christopher Roche, Gainesville, FL (US); Matt Hamilton, Gainesville, FL (US); Dean Hutchinson, Fleming Island, FL (US); Phong Diep, Gainesville, FL (US)

(73) Assignee: Exactech, Inc., Gainesville, FL (US)

(21) Appl. No.: 13/368,019

(22) Filed: Feb. 7, 2012

Related U.S. Application Data
(60) Provisional application No. 61/440,185, filed on Feb. 7, 2011.

(51) Int. Cl. A61F 2/40

(52) U.S. Cl. 623/19.11

(57) ABSTRACT

Adjustable reverse shoulder prostheses are disclosed herein. A glenoid assembly includes a glenoid plate configured for fixation to a glenoid bone for a reverse shoulder prosthesis; a glenosphere configured for connection to the glenoid plate; and an adjustment plate, wherein the adjustment plate has a connection for directly engaging the glenosphere, and wherein the adjustment plate has an articulation for directly engaging the glenoid plate at a variable angular orientation. During a reverse total shoulder arthroplasty method, a glenoid plate is fixated to a glenoid bone; an adjustment plate, configured for interfacing with both the glenoid plate and a glenosphere, is locked to the glenoid plate, wherein the adjustment plate is configured for angular orientation or positional change relative to the glenoid plate; a glenosphere is connected to the adjustment plate; and an angular orientation and position of the glenosphere relative to the fixated glenoid plate is independently adjusted.

### RELATED APPLICATIONS

This application claims the benefit of and priority to U.S. Provisional Application Ser. No. 61/440,185, filed Feb. 7, 2011, the entirety of which is hereby incorporated herein by reference for the teachings therein.

### BACKGROUND

Charles Neer coined the term cuff tear arthropathy in 1972 to describe the arthritic, eroded/collapsed condition of the glenohumeral joint following prolonged/progressive subacromial impingement resulting from massive, full thickness rotator cuff tears. This pathology is conventionally associated with extreme pain and near complete loss of function. Cuff tear arthropathy (CTA) has been historically treated with acromioplasty, arthroscopic debridement, tendon transfers, humeral tuberoplasty, arthrodesis, total shoulder arthroplasty (constrained, semi-constrained, or unconstrained), bipolar shoulder arthroplasty, hemiarthroplasty (with and without acromial spacers), and most recently (and most successfully) reverse shoulder arthroplasty. The reverse/inverse shoulder was first conceived by Neer in the early 1970's to treat patients suffering from CTA; specifically, this device was intended to provide pain relief and prevent progressive acromial, coracoid, and glenoid erosion by resisting humeral head superior migration. This was theoretically accomplished by inverting the male and female ball and socket so that the glenoid component was now convex and the humerus now concave; doing so, created a physical stop that prevents the humerus from migrating superiorly.

### SUMMARY

Adjustable reverse shoulder prostheses are disclosed herein. According to aspects illustrated herein, in an embodiment a glenoid assembly of the present disclosure includes a glenoid plate configured for fixation to a glenoid bone for a reverse shoulder prosthesis; a glenosphere configured for connection to the glenoid plate; and an adjustment plate, wherein the adjustment plate has a connection for directly engaging the glenosphere, and wherein the adjustment plate has an articulation for directly engaging the glenoid plate at a variable angular orientation. In an embodiment, the glenoid assembly is a component of an adjustable reverse total shoulder prosthesis. In an embodiment, when the glenoid assembly is implanted in a glenoid bone of a patient, a position/orientation of the glenosphere is adjustably adapted relative to the glenoid plate so as to position the glenosphere in a position that simulates native glenoid version and native glenoid inclination and/or position the glenosphere in a location that optimizes soft tissue tensioning, stability, and range of motion and minimizes impingement.

According to aspects illustrated herein, in an embodiment a glenoid assembly of the present disclosure includes a glenoid plate configured for fixation to a glenoid bone for a reverse shoulder prosthesis, wherein the glenoid plate includes a body portion, and wherein the body portion includes a plurality of through holes; at least one bone through-growth cage positioned through one of the through holes; and a glenosphere configured for connection to the glenoid plate. In an embodiment, the glenoid assembly further comprises an adjustment plate, wherein the adjustment plate has a connection for directly engaging the glenosphere, and wherein the adjustment plate has an articulation for directly engaging the glenoid plate at a variable angular orientation. In an embodiment, the glenoid assembly is a component of an adjustable reverse total shoulder prosthesis. In an embodiment, when the glenoid assembly is implanted in a glenoid bone of a patient, a shape, size, number, location, and orientation of modular fixation structures that secure the glenoid plate to an articular surface of the glenoid bone is adjustably adapted based upon a specific type of glenoid wear or malformation that the patient has in order to maximize potential for long-term glenoid fixation.

According to aspects illustrated herein, in an embodiment a reverse total shoulder arthroplasty method includes fixating a glenoid plate to a glenoid bone; locking an adjustment plate, configured for interfacing with both the glenoid plate and a glenosphere, to the glenoid plate, wherein the adjustment plate is configured for angular orientation or positional change relative to the glenoid plate; connecting a glenosphere to the adjustment plate; and independently adjusting an angular orientation and position of the glenosphere relative to the fixated glenoid plate.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 is a table showing a selection of recently reported incidence rate of scapular notching with reverse shoulder arthroplasty.

FIG. 2 shows the Boileau et al. Classifications for Glenoid Wear Patterns.

FIGS. 3A and 3B show schematic illustrations of the tendency for anterior perforation in posteriorly worn glenoids due to articular surface no longer being perpendicular to the long-axis of the scapula. The top image depicts the central stem of a glenoid implant aligning with the long axis of the scapula when implanted in a normal glenoid. The bottom image depicts the central stem of a glenoid implant perforating the anterior bone of the scapula when it is implanted in a posteriorly worn glenoid.

FIGS. 4A-4C show three views of an embodiment of an assembled reverse shoulder prosthesis of the present disclosure (oriented in a neutral position). Some of the main components of the reverse shoulder prosthesis include a humeral assembly (a humeral stem, a humeral adapter tray, and a humeral liner) and an adjustable glenoid assembly (a glenosphere, an adjustment plate, a locking ring, and a glenoid plate).

FIGS. 5A-5C show three views of an embodiment of a glenosphere of an adjustable glenoid assembly for use with a reverse shoulder prosthesis of the present disclosure.

FIGS. 6A-6D show four views of an embodiment of a glenoid plate (without compression screws) of an adjustable glenoid assembly for use with a reverse shoulder prosthesis of the present disclosure.

FIGS. 7A-7D show four views of an embodiment of a locking ring of an adjustable glenoid assembly for use with a reverse shoulder prosthesis of the present disclosure.

FIGS. 8A-8C show components of an embodiment of a glenoid assembly (a glenosphere and an adjustment plate are not illustrated) for use with a reverse shoulder prosthesis of the present disclosure.

FIGS. 9A-9C show three views of an embodiment of an adjustment plate of an adjustable glenoid assembly for use with a reverse shoulder prosthesis of the present disclosure.

FIGS. 10A-10C shows three views of an embodiment of an assembled adjustable glenoid assembly (oriented in a neutral position) for use with a reverse shoulder prosthesis of the present disclosure.

FIGS. 11A and 11B show two views of an embodiment of the assembled adjustable glenoid assembly (oriented superiorly to inferiorly by 12° in each direction; 24° total) for use with a reverse shoulder prosthesis of the present disclosure.

FIGS. 12A and 12B show two views of an embodiment of the assembled adjustable glenoid assembly (oriented anteriorly to posteriorly by 12° in each direction; 24° total) for use with a reverse shoulder prosthesis of the present disclosure.

FIGS. 13A-13C show three views of an embodiment of an adjustment plate of an adjustable glenoid assembly for use with a reverse shoulder prosthesis of the present disclosure. The taper in the adjustment plate is offset from the spherical bore.

FIGS. 14A-14D show four views of an embodiment of a glenoid plate (with central stem) of an adjustable glenoid assembly for use with a reverse shoulder prosthesis of the present disclosure.

FIGS. 15A-15D show four views of an embodiment of a glenoid plate (without central stem) of an adjustable glenoid assembly for use with a reverse shoulder prosthesis of the present disclosure.

FIGS. 16A-16D show four views of the glenoid plate of FIG. 14 with modular fixation structures for use with a reverse shoulder prosthesis of the present disclosure.

FIGS. 17A-17D show four views of the glenoid plate of FIG. 15 with modular fixation structures and locking cap screws for use with a reverse shoulder prosthesis of the present disclosure.

FIGS. 18A and 18B show two views of the glenoid plate of FIG. 17 depicting a method of connection of the modular fixation structures for use with a reverse shoulder prosthesis of the present disclosure.

FIGS. 19A-19C show three views of an embodiment of a glenoid plate (without compression screws and with central stem) of an adjustable glenoid assembly having a superior augment for use with a reverse shoulder prosthesis of the present disclosure.

FIGS. 20A-20D show four views of an embodiment of a glenoid plate (without compression screws and with central stem) of an adjustable glenoid assembly having a posterior augment for use with a reverse shoulder prosthesis of the present disclosure

FIGS. 21A and 21B show components of an embodiment of a glenoid assembly (a glenosphere and an adjustment plate are not illustrated) for use with a reverse shoulder prosthesis of the present disclosure.

FIGS. 22A-22C show schematic illustrations of the use of an embodiment of a reverse shoulder prosthesis of the present disclosure having an adjustable glenoid assembly to correct glenoid inclination for a superiorly worn glenoid (top image depicts adjustable glenoid plate when oriented in neutral position on a normal glenoid; middle image depicts adjustable glenoid plate when oriented in neutral position on a superiorly worn glenoid (not superior tilt of glenosphere due to superiorly worn bone condition); bottom image depicts adjustable glenoid plate when oriented inferiorly by 12 degrees on a superiorly worn glenoid).

FIGS. 23A-23C show schematic illustrations of the use of an embodiment of a reverse shoulder prosthesis of the present disclosure having an adjustable glenoid assembly to correct glenoid version for a posteriorly worn glenoid. (top image depicts adjustable glenoid when oriented in neutral position on a normal glenoid; middle image depicts adjustable glenoid when oriented in neutral position on a posteriorly worn glenoid; bottom image depicts adjustable glenoid when oriented anteriorly by 12 degrees on a posteriorly worn glenoid.) Note that the anteriorly oriented component corrects the positioning of the humeral component so that it is oriented with the long axis of the scapula (as in the top image).

FIGS. 24A-24C show schematic illustrations of the use of an embodiment of a reverse shoulder prosthesis of the present disclosure having an adjustable glenoid assembly to position the cage pegs to a location that does not perforate the anterior glenoid (as seen in FIG. 3) in a posteriorly worn glenoid (top image depicts adjustable glenoid when oriented in neutral position on a normal glenoid; middle image depicts adjustable glenoid when oriented in neutral position on a posteriorly worn glenoid; bottom image depicts adjustable glenoid when oriented anteriorly by 12 degrees on a posteriorly worn glenoid and with the modular central peg moved to the posterior position of the glenoid plate so that the anterior scapula is not perforated.) Note that in the bottom image the posteriorly positioned cage pegs do not perforate the anterior scapula in the posteriorly worn glenoid.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention are intended to be illustrative, and not restrictive. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

In an embodiment, the present disclosure relates to intraoperatively adjustable reverse shoulder glenoid prostheses and methods to perform reverse total shoulder arthroplasty. As used herein, the term “intraoperatively adjustable” refers to the ability of a surgeon to independently adjust both the angular orientation and position of the glenoid component used to resurface the scapula (e.g. the glenosphere). Specifically, a surgeon is able to adjust the center of rotation, version, inclination, or both angles so that the surgeon can intraoperatively orient/position the glenosphere in a desired position that maximizes stability (by balancing the soft tissue), minimizes impingement, and maximizes range of motion.

In an embodiment, the present disclosure relates to adjustable reverse shoulder glenoid prostheses and methods to perform reverse total shoulder arthroplasty in which a surgeon is able to intraoperatively adjust the size, shape, number, location, and orientation of the glenoid fixation surfaces (e.g. pegs and screws of various shapes, sizes, and surface finishes) based upon the patient's particular type of glenoid wear. Specifically, the surgeon is able to adjust the shape, size, number, location, and orientation of the central stem bone cages or similar fixation structures used to ensure long-term glenoid fixation to the glenoid plate.

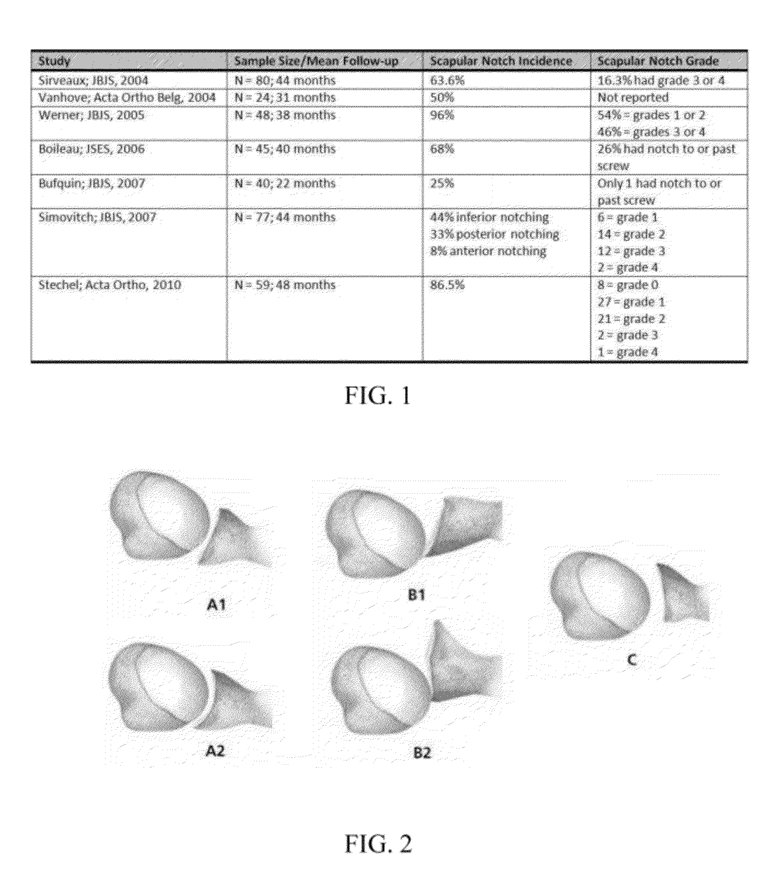

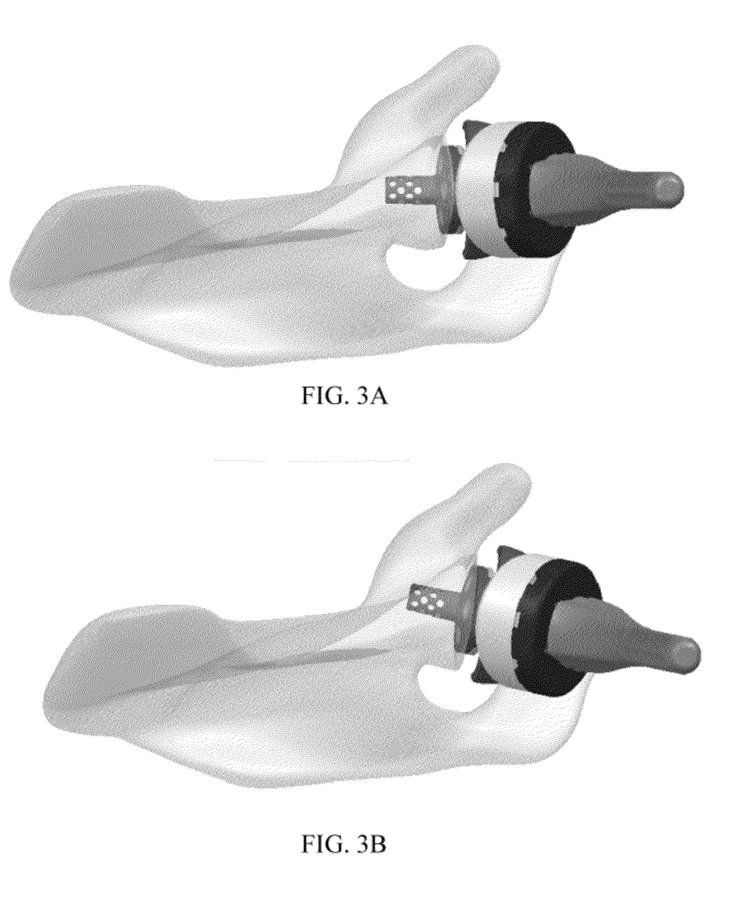

Some currently available reverse shoulder prostheses are associated with a number of different types of complications including, but not limited to, glenoid loosening, scapular “notching” (more descriptively called inferior glenoid erosion), acromion fractures, dislocation (head from poly and poly insert from humeral stem), instability, humeral stem fracture, humeral stem loosening, and glenoid screw fracture. Scapular notching is a unique complication of some currently available reverse shoulder prostheses. Predictors of scapular notching include, but are not limited to, surgical approach, glenoid wear, preoperative diagnosis, infraspinatus muscle quality, cranial-caudal positioning, and tilt of the glenosphere. FIG. 1 is a table showing a selection of recently reported incidence rate of scapular notching with reverse shoulder arthroplasty. A classification system for posterior glenoid wear described by Boileau et al is presented in FIG. 2. Posterior glenoid wear (such as that depicted in FIG. 2) often results in inadequate fixation due to the following non-limiting reasons:
1) Nonanatomic loading patterns
2) Decreased bone stock (specifically, the implant seats on a cancellous bone bed rather than a cortical bone bed or it sits on a combination of cortical anteriorly and cancellous posteriorly which can lead to subsidence since the cancellous bone is ˜15 to 20 times less dense/strong than the cortical bone)
3) Nonideal placement of the fixation surfaces (specifically, the screws or pegs often perforate the anterior scapula since perpendicular to the posteriorly worn glenoid face is no longer in-line with the long axis of the scapula as depicted in FIGS. 3A and 3B or due to impartial or incomplete seating of the implant since the bone which it sits may have multiple concave surfaces)

Various embodiments of the present invention are directed to reverse shoulder prostheses that have benefits including, but not limited to, 1) lengthen/tension deltoid to improve muscle efficiency; 2) maintain center of rotation on (or close to) the glenoid fossa to minimize the effective moment arm; and/or 3) invert the concavities of the natural joint to create a physical stop to prevent humeral head superior migration. The complications/concerns that various embodiments of the present invention may minimize include, but are not limited to, 1) reduce the incidence of impingement; 2) reduce the incidence of scapular notching; 3) improve stability and range of motion (ROM); 4) decrease the incidence of dislocation; 5) improve glenoid fixation; 6) conserve bone (particularly in the instance of a worn glenoid in which the worn glenoid would often need to be eccentrically reamed to correct version or tilt); and/or 7) better facilitate a conversion of a hemi- or total shoulder to a reverse shoulder. Range of Motion (ROM) is defined as the humeral rotation occurring between inferior and superior impingement, wherein inferior and superior impingement are defined as the point where the humeral liner extends past the glenosphere.

In an embodiment, the present disclosure relates to intraoperatively adjustable reverse shoulder prostheses which may have the following non-limiting benefits: a reverse shoulder prosthesis of the present disclosure may be integrated with a primary system (total shoulder prostheses) and may retain the primary stem for revision; a reverse shoulder prosthesis of the present disclosure may use existing humeral implant inventory, existing humeral instrumentation, and/or a similar surgical technique; a reverse shoulder prosthesis of the present disclosure may be associated with an increase in ROM (as compared to some currently available prosthesis); a reverse shoulder prosthesis of the present disclosure may be associated with a reduction in the incidence of scapular notching (i.e. medial/inferior impingement of humerus on scapula) as a result of the reduction in neck angle from 155° to 145° (as compared to some currently available prosthesis) and the increase in humeral liner size (since the liner may be brought out of the proximal humerus; a reverse shoulder prosthesis of the present disclosure may maintain the low incidence of glenosphere loosening by utilizing a glenosphere/screw/baseplate design; a reverse shoulder prosthesis of the present disclosure may include a glenoid plate having a bone “through-growth” cage designed to enhance fixation; a reverse shoulder prosthesis of the present disclosure may include a glenoid plate that allows for the insertion of a compression screw (e.g., at up to 15 degrees of angular variability) in any of the holes to maximize bone purchase; and a reverse shoulder prosthesis of the present disclosure may include a glenoid plate that allows the use of a locking cap screw which can be attached to any compression screw thereby making each screw a locking/compression screw.

In an embodiment, the present disclosure relates to intraoperatively adjustable reverse shoulder prostheses and methods for implanting the prosthesis that enables a surgeon to secure the glenoid plate directly to the glenoid bone and then adjustably adapt the position/orientation of the glenosphere relative to the glenoid plate in order to position the glenosphere that simulates native glenoid version and native glenoid inclination and/or position the glenosphere in a location that optimizes soft tissue tensioning, stability, and range of motion and minimizes impingement. It should be noted that in the absence of such an adjustable prosthesis, the surgeon would eccentrically ream to correct the version of inclination, where eccentrically reaming the glenoid means that they are reaming away the nonworn (often cortical) glenoid bone. Thus, the adjustable prostheses disclosed herein are bone conserving. The adjustable reverse shoulder prostheses disclosed herein include an adjustment plate which has a locking spherical articulation with the glenoid plate (e.g. the component that fixes directly to the bone with locking/compression screws and a pressfit male boss) and a taper connection to the glenosphere (e.g. the convex component that articulates to the concave humeral liner in reverse shoulder arthroplasty). The mating spherical surfaces between the adjustment plate and the glenoid plate enables a surgeon to position the adjustment plate at a variable angular orientation. As used herein, the term “variable angular orientation” means that a surgeon can intraoperatively adjust or change the angular orientation of the adjustment plate relative to the glenoid plate. In an embodiment, the mating spherical surfaces between the adjustment plate and the glenoid plate enables a surgeon to position the adjustment plate at a variable angular orientation of about ±12°. The adjustment plate may also be offset or lengthened to increase the degree of adjustability in order to optimize soft tissue tensioning which may be useful to modify the center of rotation in the cases of bone or soft tissue deficiencies in order to better tension the joint, for example when performing a reverse shoulder to treat a complex proximal humeral fracture or to treat a severely eroded glenoid bone/scapula.

In an embodiment, the present disclosure relates to reverse shoulder prostheses and methods for implanting the prosthesis that enables a surgeon to secure the glenoid plate directly to the glenoid bone (or malformed/eroded glenoid bone) and then adjustably adapt the shape, size, number, location, and orientation of the glenoid fixation structures that secure the glenoid plate to the glenoid articular surface based upon the specific type of glenoid wear or malformation that the patient may have in order to maximize the potential for long-term glenoid fixation. The reverse shoulder prostheses of the present disclosure include modular fixation structures which connect to the glenoid plate in multiple different locations and orientations. These modular fixation structures can be secured via taper connections, with screws, or both. Additionally, these modular fixation structures can be of varying size, shape, material, and surface finish to ensure that the glenoid plate is optimally secured to the patient's bone. The method of securing these modular long-term glenoid fixation structures is useful when the surgeon chooses to use bone graft (for example, grafting the superior portion of the glenoid in a patient with cuff tear arthropathy, grafting the inferior portion of the glenoid due to prior scapular notching, or grafting the posterior side of the glenoid due to posterior wear); the position and orientation of placement of the modular fixation structures can bridge the graft and increase the probability that the graft incorporates. Additionally, this method of securing these modular long-term glenoid fixation structures is useful when a surgeon chooses to eccentrically ream the glenoid in order to restore the native glenoid angulation (for example, to eccentrically ream the anterior side of the glenoid in order to restore the native glenoid version); the position and orientation of placement of the modular fixation structures can ensure that the fixation surface does not perforate the opposing cortical shell of the scapula, compromising fixation.

A glenosphere component of a reverse shoulder prosthesis of the present disclosure is modularly connected to the adjustment plate (via a taper connection) and can be used interchangeably with different sizes of glenospheres (e.g. diameters and/or thicknesses) to ensure that the position/orientation of the glenosphere is optimized for each patient in terms of optimal soft tissue tensioning, maximal stability and range of motion, and minimal impingement. Similarly, the adjustment plate is modularly connected to the glenoid plate (via a spherical mating surface and a locking screw) and can be used interchangeably with different sizes of adjustment plates (e.g. angled, offset, or adjustment plates of varying thicknesses) to ensure that the position/orientation of the glenosphere is optimized for each patient in terms of optimal soft tissue tensioning, maximal stability and range of motion, and minimal impingement. Similarly, the glenoid plate can be shaped, angled, or augmented (e.g. posteriorly, superiorly, etc) in such a way to optimize fixation for each patient's glenoid morphology and help to restore native glenoid version and native glenoid inclination intraoperatively should the surgeon desire. For example, if a patient has a type C glenoid wear according to the classification system described in FIG. 2 (e.g. retroversion>25°) then a 13° posteriorly augmented glenoid plate may be used in combination with the adjustment plate angled in the anterior direction by 12° in order to bring the version of the glenoid back to neutral (thus, the adjustable reverse shoulder prevented the eccentric reaming of approximately 12° of bone to correct the deformity).

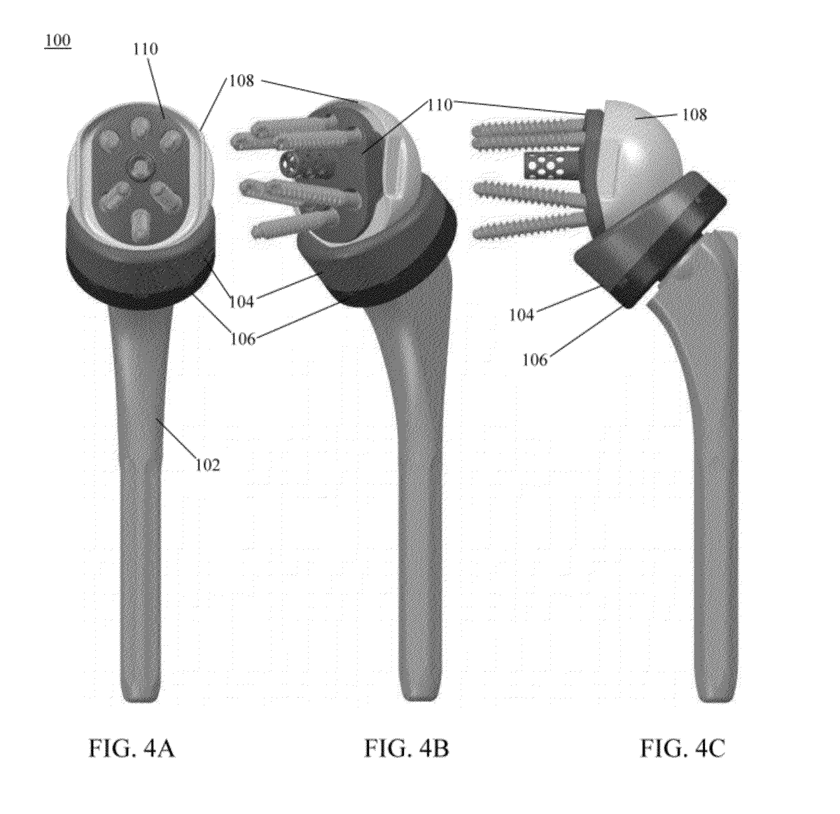

FIGS. 4A-4C show three views of an embodiment of an assembled adjustable reverse shoulder prosthesis 100 of the present disclosure when the adjustable glenoid is positioned in its neutral orientation. The components of the reverse shoulder prosthesis include a humeral stem 102 (which may be used in pressfit and/or cemented applications and may be constructed from titanium), a humeral liner 104 (a concave component which mates with the convex glenosphere; may be constructed from UHMWPE), a humeral adapter tray 106 (which connects the humeral liner to the humeral stem; may be constructed from titanium), a glenosphere 108 (may be constructed from cobalt chrome), an adjustment plate (may be constructed from titanium, not visible in FIGS. 4A-4C), a locking ring (may be constructed from titanium, not visible in FIGS. 4A-4C) and a glenoid plate 110 (may be constructed from titanium). Although FIGS. 4A-4C show glenoid plate 110, which will be described in more detail below, it should be noted that any of the glenoid plates of the present disclosure can replace glenoid plate 110 in the adjustable reverse shoulder prosthesis 100. A number of screws and fixations devices for assembly of the individual components to one another and for assembly of the construct to the native bone are illustrated (all may be constructed from titanium). In an embodiment, to reduce the incidence of impingement and scapular notching, the neck angle of the reverse shoulder prosthesis may be reduced from 155° to 145°. In an embodiment, the humeral liner 104 may be manufactured from Connection GXL (i.e. enhanced poly) and may utilize a “mushroom” apical-locking mechanism to attach the humeral liner 104 to the humeral adapter tray 106, therefore, a low incidence of humeral liner wear and disassociation may be expected.

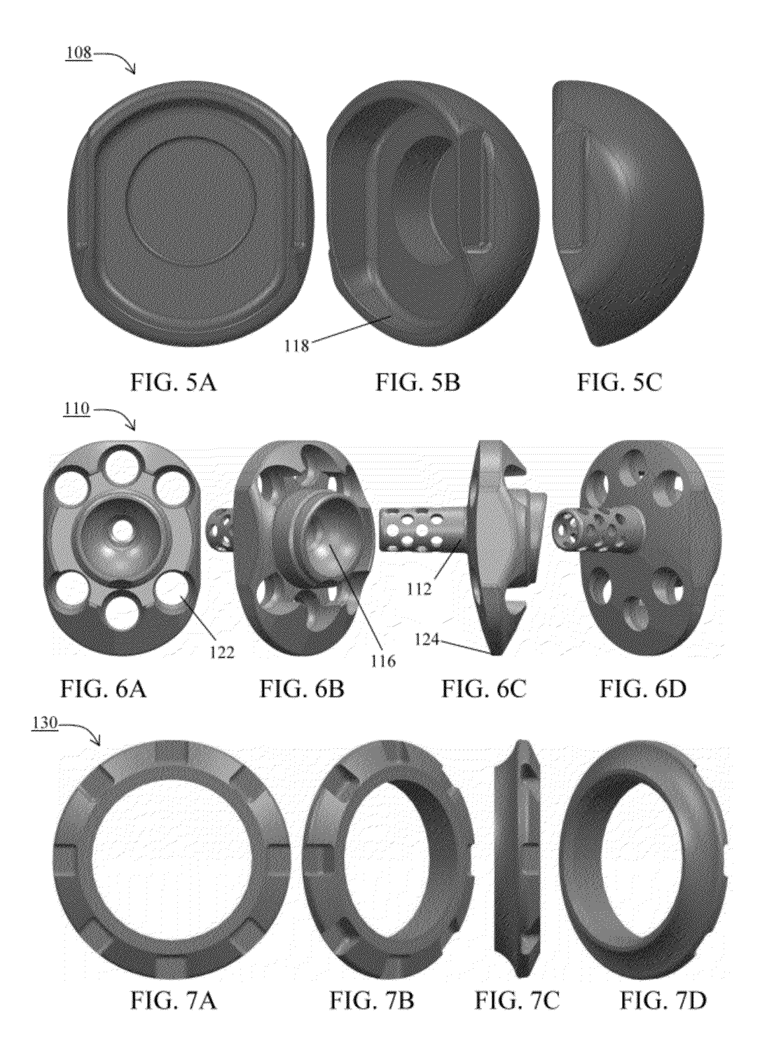

FIGS. 5A-5C show three views of the glenosphere 108 of FIGS. 4A-4C. The glenosphere 108 is a convex component that articulates to the concave humeral liner in reverse shoulder arthroplasty. The glenosphere 108 is modularly connected to an adjustment plate of the present disclosure (via a taper connection). The glenosphere body is at least partially hollow, having a central recessed area for accepting a mating portion of the adjustment plate and a bottom recessed track area 118 for which a bottom surface of the adjustment plate sits. In an embodiment, the glenosphere body has a perimeter shape that is generally elongated along a superior-inferior axis. In an embodiment, the glenosphere body has a perimeter shape that is generally elongated along a superior-inferior axis to substantially match a perimeter shape of the glenoid plate. In an embodiment, the glenosphere body has a perimeter shape that is generally circular. In an embodiment, the glenosphere body has a perimeter shape that is generally circular to substantially match a perimeter shape of the glenoid plate. In an embodiment, the anterior and posterior sides of the glenosphere may be chamfered; thereby, allowing the glenosphere to be inserted into the wound site and sit flush on the resected surface without having to remove any excess glenoid bone.

FIGS. 6A-6D show four views of the glenoid plate 110 (without compression screws) of FIGS. 4A-4C. In an embodiment, the glenoid plate 110 is substantially oval in shape. The glenoid plate 110 fixes directly to the glenoid bone (or malformed/eroded glenoid bone) with locking/compression screws and a pressfit male boss. The glenoid plate 110 can be shaped, angled, or augmented (e.g. posteriorly, superiorly, etc) in such a way to optimize fixation for each patient's glenoid morphology and help to restore native glenoid version and native glenoid inclination intraoperatively should a surgeon desire. The glenoid plate 110 includes a spherical tapered surface 116 that mates with a locking spherical articulation of an adjustment plate of the present invention. The glenoid plate 110 includes a body portion having a front and a back, and a central stem 112 that is a superiorly shifted fixed structure. In order to conserve the much needed glenoid bone, the glenoid plate 110 according to an embodiment is designed so that the central stem 112 (also referred to herein as the “stem portion” or “cage”) is shifted superiorly (e.g., by 4 mm)—enabling a surgeon to maintain the traditional surgical technique with the reverse as would be performed for total shoulder arthroplasty (i.e. drilling a hole in the center of the glenoid bone where the defect would occur; thereby, conserving bone). The body portion of the glenoid plate 110 has a vertical dimension, wherein a central point of the vertical dimension divides the body portion into an upper half and a lower half, wherein the central stem 112 has a central longitudinal axis, and wherein the central stem 112 extends from the body portion from a position on the body portion such that the central longitudinal axis of the central stem 112 is sufficiently superiorly shifted from the central point of the vertical dimension so that when the glenoid plate 110 is implanted in a glenoid bone so that the central stem 112 is positioned in a center of the glenoid bone, a distal rim of the glenoid plate 110 is aligned with a distal edge of an articular surface of the glenoid bone. In an embodiment, the glenoid plate hole 122 positions are designed to allow conversion of a traditional peg and keel glenoid. In some embodiments, in order to improve glenoid fixation, the central stem 112 allows bone “through-growth” using inductive/conductive bone graft. Bone graft can be placed into the stem 112 prior to securing the plate with screws and/or after (e.g., by injecting the graft through a syringe in the top of the plate). The bone through-growth fixation stem 112 can be either cylindrical (e.g., to revise a peg glenoid) or non-cylindrical (e.g., to revise a keel glenoid). In some embodiments, the glenoid plate 110 of the present disclosure may incorporate several other features which should work to conserve glenoid bone and/or improve fixation. The glenoid plate 110 may have a curved-back to minimize the amount of bone removed for implantation, (compared to the flat-back glenoid plate designs, as the native glenoid bone is also curved). Additionally, one or more screw holes in the glenoid plate may have a female spherical feature which mates with the male spherical head of the compression screw. Doing so may allow for each compression screw to be angled/oriented in any desired direction—thereby improving the possibility of screw purchase.

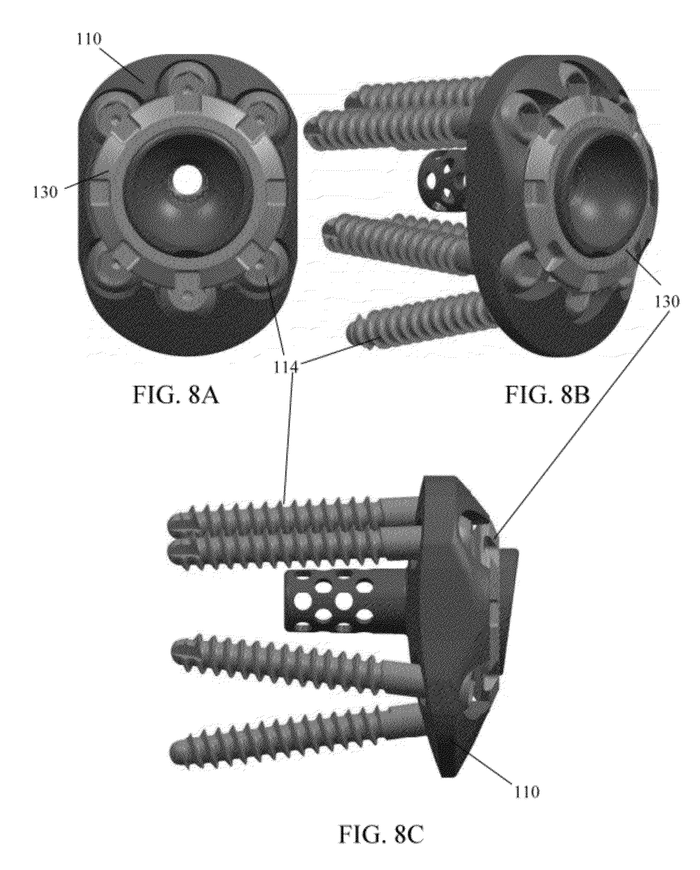

FIGS. 7A-7D show four views of an embodiment of a locking ring 130 for attachment to a glenoid plate of the present invention. As illustrated in FIGS. 8A-8C, the locking ring 130 is used to secure polyaxial compression screws 114 (which, in an embodiment, can be inserted into the glenoid/scapula at approximately 8°) to the glenoid plate 110 and to prevent the polyaxial compression screws 114 from backing out (e.g. losing their compression). The locking ring 130 works by threading directly to a glenoid plate of the present invention, and the locking ring 130 secures/locks all the polyaxial compression screws 114 at the same time. The polyaxial compression screws 114 can range in length between about 18 mm to about 54 mm.

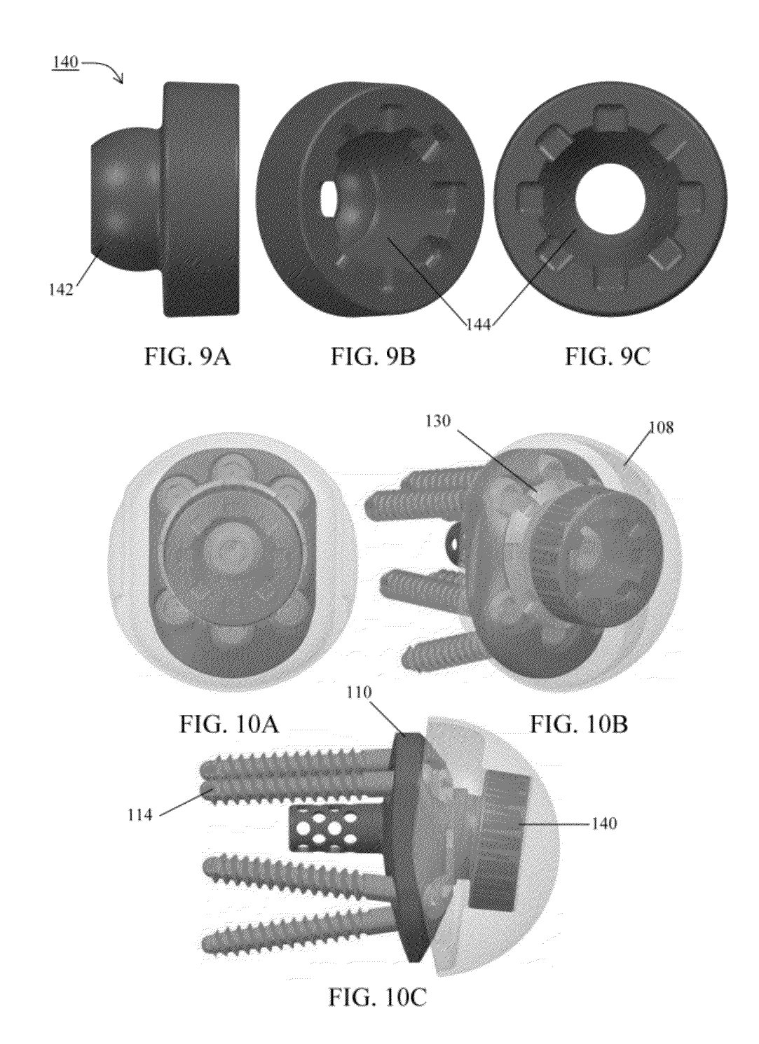

FIGS. 9A-9C show three views of an embodiment of an adjustment plate 140 of the present invention. The adjustment plate 140 has a locking articulation 142 with the glenoid plate 110 and a connection 144 to a glenosphere. In an embodiment, the locking articulation 142 is spherical in shape. In an embodiment, the connection 144 is tapered. The articulation 142 permits the adjustment plate 140 to be secured to a glenoid plate of the present disclosure at a variable angular orientation/position. The adjustment plate 140 can be used interchangeably with different sizes of glenospheres (e.g. diameters and/or thicknesses) to ensure that the position/orientation of the glenosphere is optimized for each patient in terms of optimal soft tissue tensioning, maximal stability and range of motion, and minimal impingement. The body portion of the adjustment plate 140 has a front face, a back face in which the locking articulation 142 extends from, and a thickness therebetween. The body portion of the adjustment plate 140 has a central horizontal axis and a central vertical axis. The central horizontal axis and the central vertical axis of the adjustment plate 140 intersect at a central point that divides the body portion into an upper half, a lower half, a right half and a left half.

The articulation 142 permits the adjustment plate 140 to be secured to a glenoid plate of the present disclosure at a variable angular orientation/position as depicted in FIGS. 10-12. The adjustment plate 140 is modularly connected to a glenoid plate (via the articulation 142 and a locking screw), and the glenoid plate can be used interchangeably with different sizes of adjustment plates (e.g. angled, offset, or adjustment plates of varying thicknesses) to ensure that the position/orientation of the glenosphere is optimized for each patient in terms of optimal soft tissue tensioning, maximal stability and range of motion, and minimal impingement.

FIGS. 10A-10C shows three views of an embodiment of an assembled adjustable glenoid assembly (oriented in a neutral position) for use with a reverse shoulder prosthesis of the present disclosure. Although the adjustable glenoid assembly of FIGS. 10A-10C show the assembly oriented in a neutral position, in an embodiment, the assembly can be oriented such that the adjustment plate/glenosphere is inferiorly biased at 7.5°. The assembled adjustable glenoid assembly includes the glenosphere 108, the glenoid plate 110, polyaxial compression screws 114, the locking ring 130 and the adjustment plate 140. The polyaxial compression screws 114 may include a spherical head which enables the screw to be angularly oriented within the glenoid plate 110 (e.g., up to 17.5 degrees) in any desired direction—in one specific example, the holes in glenoid plate 110 may have corresponding concavities. The adjustment plate 140 indirectly connects the glenosphere 108 to the glenoid plate 110. As used herein, the term “indirectly connects” refers to the non-direct connection of the glenosphere 108 to the glenoid plate 110. The locking ring 130 works by threading directly to the glenoid plate 110, and the locking ring 130 secures/locks all the polyaxial compression screws 114 at the same time.

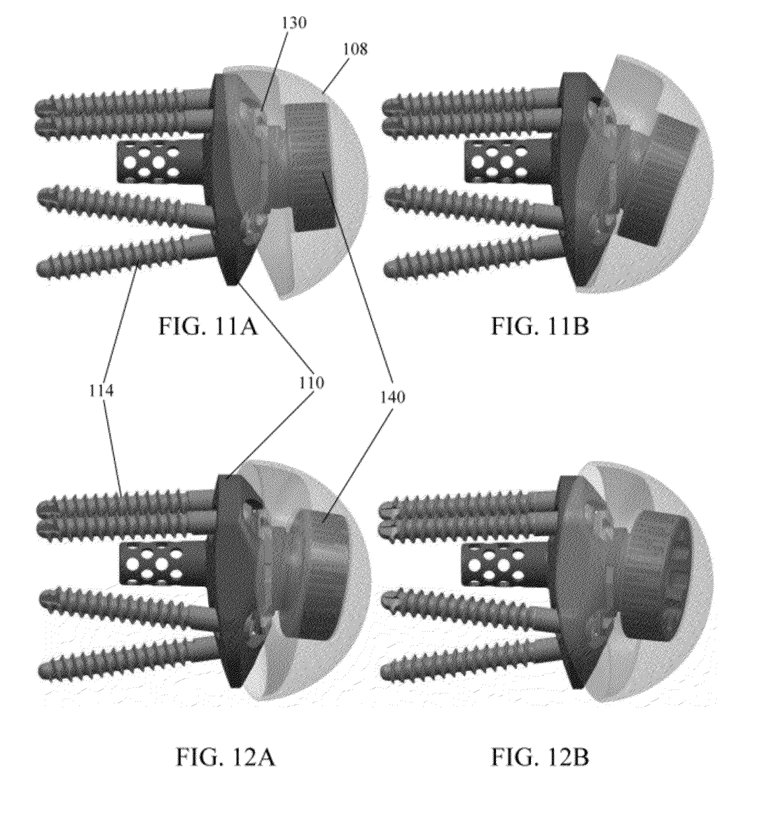

FIGS. 11A and 11B show two views of an embodiment of the assembled adjustable glenoid assembly (oriented superiorly to inferiorly by 12° in each direction; 24° total) for use with a reverse shoulder prosthesis of the present disclosure. FIGS. 12A and 12B show two views of an embodiment of the assembled adjustable glenoid assembly (oriented anteriorly to posteriorly by 12° in each direction; 24° total) for use with a reverse shoulder prosthesis of the present disclosure.

FIGS. 13A-13C show three views of an embodiment of an adjustment plate 240 of an adjustable glenoid assembly for use with a reverse shoulder prosthesis of the present disclosure. The adjustment plate 240 has an offset locking articulation 242 for engaging the glenoid plate and an offset connection 244 to a glenosphere. In an embodiment, the locking articulation 242 is spherical in shape. In an embodiment, the connection 244 is tapered. The articulation 242 permits the adjustment plate 240 to be secured to a glenoid plate of the present disclosure at a variable angular orientation/position. The adjustment plate 240 can be used interchangeably with different sizes of glenospheres (e.g. diameters and/or thicknesses) to ensure that the position/orientation of the glenosphere is optimized for each patient in terms of optimal soft tissue tensioning, maximal stability and range of motion, and minimal impingement. The body portion of the adjustment plate 240 has a front face, a back face in which the locking articulation 242 extends from, and a thickness therebetween. The body portion of the adjustment plate 240 has a central horizontal axis and a central vertical axis. The central horizontal axis and the central vertical axis of the adjustment plate 240 intersect at a central point that divides the body portion into an upper half, a lower half, a right half and a left half.

In an embodiment, the offset connection 244 permits a glenosphere to be positioned in an offset location relative to a glenoid plate. In an embodiment, the offset positioning of the glenosphere is advantageous to aid the surgeon to balance the joint in certain bone deformities or soft tissue deficiencies. In an embodiment, the offset taper connection 244 permits a glenosphere to be positioned in an offset location of about 3 mm relative to a glenoid plate. The adjustment plate 240 is modularly connected to a glenoid plate (via the spherical articulation 242 and a locking screw), and the glenoid plate can be used interchangeably with different sizes of adjustment plates (e.g. angled, offset, or adjustment plates of varying thicknesses) to ensure that the position/orientation of the glenosphere is optimized for each patient in terms of optimal soft tissue tensioning, maximal stability and range of motion, and minimal impingement.

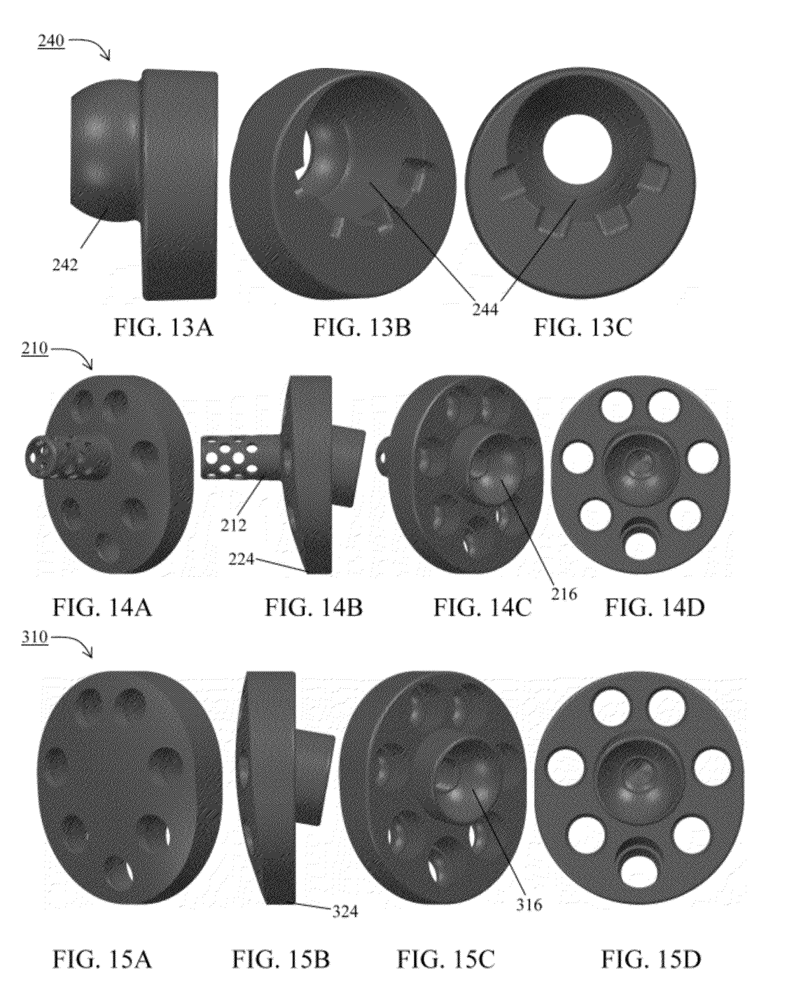

FIGS. 14A-14D and 15A-15D present various embodiments of glenoid plates 210 and 310. In an embodiment, the plates 210 and 310 are substantially oval in shape. In an embodiment, the plates 210 and 310 are substantially circular in shape. The glenoid plates 210 and 310 fix directly to the glenoid bone (or malformed/eroded glenoid bone) with locking/compression screws and a pressfit male boss. The glenoid plates 210 and 310 can be shaped, angled, or augmented (e.g. posteriorly, superiorly, etc) in such a way to optimize fixation for each patient's glenoid morphology and help to restore native glenoid version and native glenoid inclination intraoperatively should a surgeon desire. The glenoid plates 210 and 310 include a spherical tapered surface 216 and 316, respectively, that mates with a locking spherical articulation of an adjustment plate of the present invention. The glenoid plate 210 includes a body portion having a front and a back, and a central stem 212 that is a superiorly shifted fixed structure. In order to conserve the much needed glenoid bone, the glenoid plate 210 according to an embodiment is designed so that the central stem 212 is shifted superiorly (e.g., by 4 mm)—enabling a surgeon to maintain the traditional surgical technique with the reverse as would be performed for total shoulder arthroplasty (i.e. drilling a hole in the center of the glenoid bone where the defect would occur; thereby, conserving bone). The body portion of the glenoid plate 210 has a vertical dimension, wherein a central point of the vertical dimension divides the body portion into an upper half and a lower half, wherein the central stem 212 has a central longitudinal axis, and wherein the central stem 212 extends from the body portion from a position on the body portion such that the central longitudinal axis of the central stem 212 is sufficiently superiorly shifted from the central point of the vertical dimension so that when the glenoid plate 210 is implanted in a glenoid bone so that the central stem 212 is positioned in a center of the glenoid bone, a distal rim of the glenoid plate 210 is aligned with a distal edge of an articular surface of the glenoid bone. In an embodiment, the glenoid plate hole positions shown in FIGS. 14A-14D and FIGS. 15A-15D are designed to allow conversion of a traditional peg and keel glenoid. In some embodiments, in order to improve glenoid fixation, the central stem 212 allows bone “through-growth” using inductive/conductive bone graft. Bone graft can be placed into the stem prior to securing the plate with screws and/or after (e.g., by injecting the graft through a syringe in the top of the plate). The bone through-growth fixation stem 212 can be either cylindrical (e.g., to revise a peg glenoid) or non-cylindrical (e.g., to revise a keel glenoid). In some embodiments, the glenoid plates 210 and 310 shown in FIGS. 14A-14D and FIGS. 15A-15D may incorporate several other features which should work to conserve glenoid bone and/or improve fixation. The glenoid plates 210 and 310 shown in FIGS. 14A-14D and FIGS. 15A-15D may have a curved-back to minimize the amount of bone removed for implantation, (compared to the flat-back glenoid plate designs, as the native glenoid bone is also curved). Additionally, one or more screw holes in the glenoid plates 210 and 310 shown in FIGS. 14A-14D and FIGS. 15A-15D may have a female spherical feature which mates with the male spherical head of the compression screw. Doing so may allow for each compression screw to be angled/oriented in any desired direction—thereby improving the possibility of screw purchase. Additionally, one or more of the screw holes in the glenoid plates 210 and 310 shown in FIGS. 14A-14D and FIGS. 15A-15D may have a threaded feature for attachment of a locking cap. The cap screw may have a female spherical feature which compresses the spherical head of the compression screw; thereby locking it to the plate at whatever angle/orientation the screw was inserted into the bone (preventing it from backing out).

Many patients who receive a reverse shoulder prosthesis have some form of compromised glenoid bone stock due to age, deformity and/or pathology. A glenoid plate of the present disclosure enables a surgeon to secure the glenoid plate directly to the glenoid bone (or malformed/eroded glenoid bone) and then adjustably adapt the shape, size, number, location, and orientation of the glenoid fixation structures (e.g., compression screws and bone cages) that secure the glenoid plate to the glenoid articular surface based upon the specific type of glenoid wear or malformation that the patient may have in order to maximize the potential for long-term glenoid fixation. These modular fixation structures can be secured via taper connections, with screws, or both. Additionally, these modular fixation structures can be of varying size, shape, material, and surface finish to ensure that the glenoid plate is optimally secured to the patient's bone.

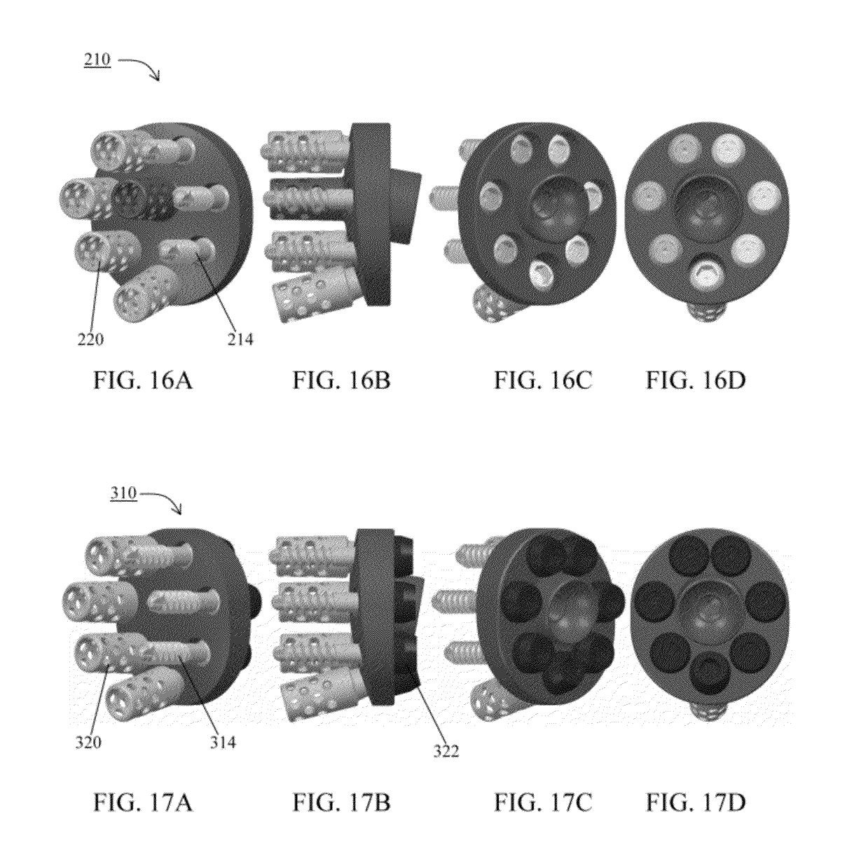

FIGS. 16A-16D show four views of the glenoid plate 210 of FIGS. 14A-14D with modular fixation structures positioned in any one or more of the holes. These modular fixation structures can be positioned in any one or more of the holes in any of the glenoid plates of the present invention. Although FIGS. 16A-16D show modular fixation structures positioned in all of the holes of the glenoid plate 210, it should be understood that the glenoid plate 210 may include one, two, three, four, five, six or seven modular fixation structures depending on the amount of fixation required. In an embodiment, the glenoid plate 210 includes modular fixation structures that are bone “through-growth” cages 220. In an embodiment, the glenoid plate 210 includes modular fixation structures that are compression screws 214. The compression screws 214 may include a spherical head which enables the screw to be angularly oriented within the glenoid plate 210 (e.g., up to 17.5 degrees) in any desired direction—in one specific example, the holes in glenoid plate 210 may have corresponding concavities. Examples of glenoid plate modular fixation structure configurations include, but are not limited to, configuration #1 in which one fixation structure is placed in the superior hole and three fixation structures in the three most inferior holes; configuration #2 in which one fixation structure is placed in the superior hole and two fixation structures in the two most inferior holes; configuration #3 in which one fixation structure is placed in the superior hole and one fixation structure in the most inferior hole; configuration #4, representing the conversion of a pegged glenoid to a reverse shoulder, in which one fixation structure is placed in the most inferior hole and three fixation structures in the three most superior holes; and configuration #5, representing the conversion of a keeled glenoid to a reverse shoulder, in which two fixation structures are placed in the two most anterior holes and two fixation structures are placed in the two most posterior holes. In an embodiment, the glenoid plate 210 includes modular fixation structures that are a combination of bone “through-growth” cages 220 and compression screws 214.

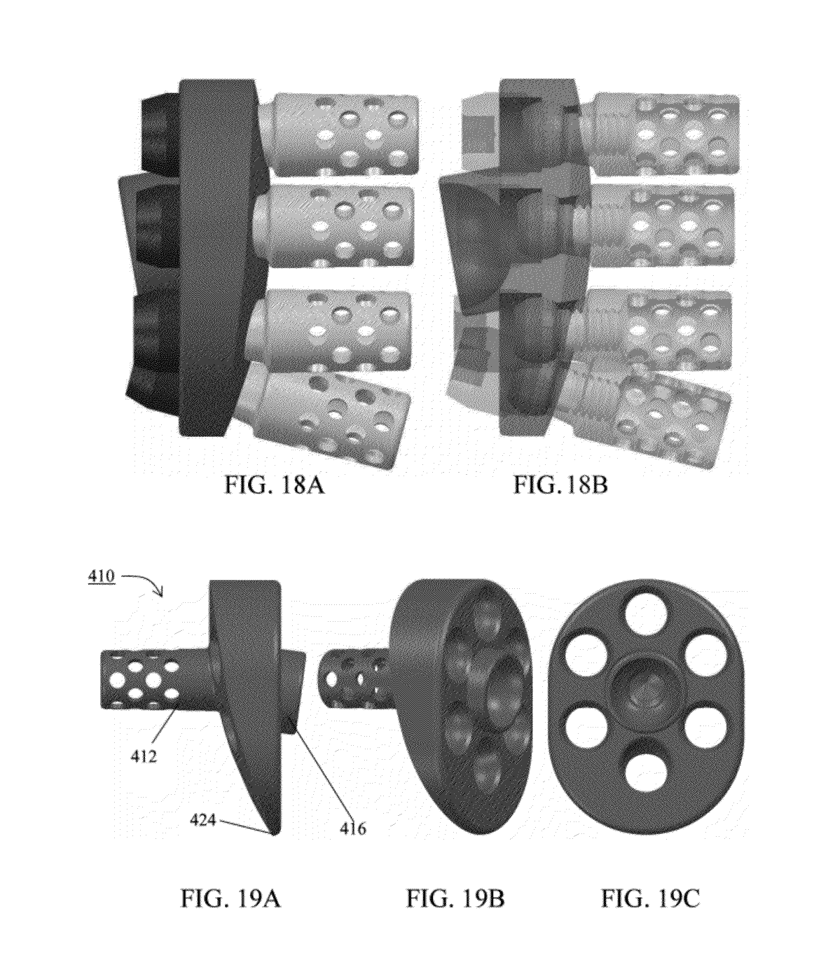

FIGS. 17A-17D show four views of the glenoid plate 310 of FIG. 15 with modular fixation structures and locking cap screws 322 positioned in any one or more of the holes. These modular fixation structures and locking cap screws can be positioned in any one or more of the holes in any of the glenoid plates of the present invention. Although FIGS. 17A-17D show modular fixation structures positioned in all of the holes of the glenoid plate 310, it should be understood that the glenoid plate 310 may include one, two, three, four, five, six or seven modular fixation structures depending on the amount of fixation required. In an embodiment, the glenoid plate 310 includes modular fixation structures that are bone “through-growth” cages 320. In an embodiment, the glenoid plate 310 includes modular fixation structures that are compression screws 314. In an embodiment, the glenoid plate 310 includes modular fixation structures that are a combination of bone “through-growth” cages 320 and compression screws 314. The compression screws 314 may include a spherical head which enables the screw to be angularly oriented within the glenoid plate 310 (e.g., up to 17.5 degrees) in any desired direction—in one specific example, the holes in glenoid plate 310 may have corresponding concavities. Examples of glenoid plate modular fixation structure configurations include, but are not limited to, configuration #1 in which one modular fixation structure is placed in the superior hole and three modular fixation structures in the three most inferior holes; configuration #2 in which one modular fixation structure is placed in the superior hole and two modular fixation structures in the two most inferior holes; configuration #3 in which one modular fixation structure is placed in the superior hole and one modular fixation structure in the most inferior hole; configuration #4, representing the conversion of a pegged glenoid to a reverse shoulder, in which one modular fixation structure is placed in the most inferior hole and three modular fixation structures in the three most superior holes; and configuration #5, representing the conversion of a keeled glenoid to a reverse shoulder, in which two modular fixation structures are placed in the two most anterior holes and two modular fixation structures are placed in the two most posterior holes. The locking cap screws 322 according to an embodiment of the present invention may be screwed into the glenoid plate 310 on top of a compression screw 314 to prevent the compression screw 314 from backing out and/or to lock the compression screw 314 in a desired angular orientation. FIGS. 18A and 18B show two views of the glenoid plate of FIG. 17 depicting a method of connection of the modular fixation structures for use with a reverse shoulder prosthesis of the present disclosure.

FIGS. 19A-19C present an embodiment of a glenoid plate 410 in which the superior portion of the glenoid plate 410 is augmented by 10°. In an embodiment, the glenoid plate 410 is substantially oval in shape. The glenoid plate 410 fixes directly to the glenoid bone (or malformed/eroded glenoid bone) with locking/compression screws and a pressfit male boss. The glenoid plate 410 can be shaped, angled, or augmented (e.g. posteriorly, superiorly, etc) in such a way to optimize fixation for each patient's glenoid morphology and help to restore native glenoid version and native glenoid inclination intraoperatively should a surgeon desire. The glenoid plate 410 includes a spherical tapered surface 416 that mates with a locking spherical articulation of an adjustment plate of the present invention. Glenoid plate 410 includes a central stem 412. The stem 412 is a superiorly shifted fixation structure. In order to conserve the much needed glenoid bone, the central stem 412 is shifted superiorly (e.g., by 4 mm)—enabling a surgeon to maintain the traditional surgical technique with the reverse as would be performed for total shoulder arthroplasty (i.e. drilling a hole in the center of the glenoid bone where the defect would occur; thereby, conserving bone). In an embodiment, the central stem 412 is sufficiently superiorly shifted from the central point of the vertical dimension so that, when the glenoid plate 410 is implanted in a glenoid bone so that the central stem 412 is positioned in a center of the glenoid bone, a distal rim 424 of the glenoid plate 410 is aligned with a distal edge of an articular surface of the glenoid bone. In an embodiment, the glenoid plate hole positions are designed to allow conversion of a traditional peg and keel glenoid. In some embodiments, in order to improve glenoid fixation, the central stem 412 allows bone “through-growth” using inductive/conductive bone graft. Bone graft can be placed into the stem prior to securing the plate with screws and/or after (e.g., by injecting the graft through a syringe in the top of the plate). The bone through-growth fixation stem 412 can be either cylindrical (e.g., to revise a peg glenoid) or non-cylindrical (e.g., to revise a keel glenoid). In some embodiments, the glenoid plate 410 may incorporate several other features which should work to conserve glenoid bone and/or improve fixation. The glenoid plate 410 may have a curved-back to minimize the amount of bone removed for implantation, (compared to the flat-back glenoid plate designs, as the native glenoid bone is also curved). Additionally, one or more screw holes in the glenoid plate 410 may have a female spherical feature which mates with the male spherical head of the compression screw. Doing so may allow for each compression screw to be angled/oriented in any desired direction—thereby improving the possibility of screw purchase. Additionally, one or more of the screw holes in the glenoid plate 410 may have a threaded feature for attachment of a locking cap. The cap screw may have a female spherical feature which compresses the spherical head of the compression screw; thereby locking it to the plate at whatever angle/orientation the screw was inserted into the bone (preventing it from backing out).

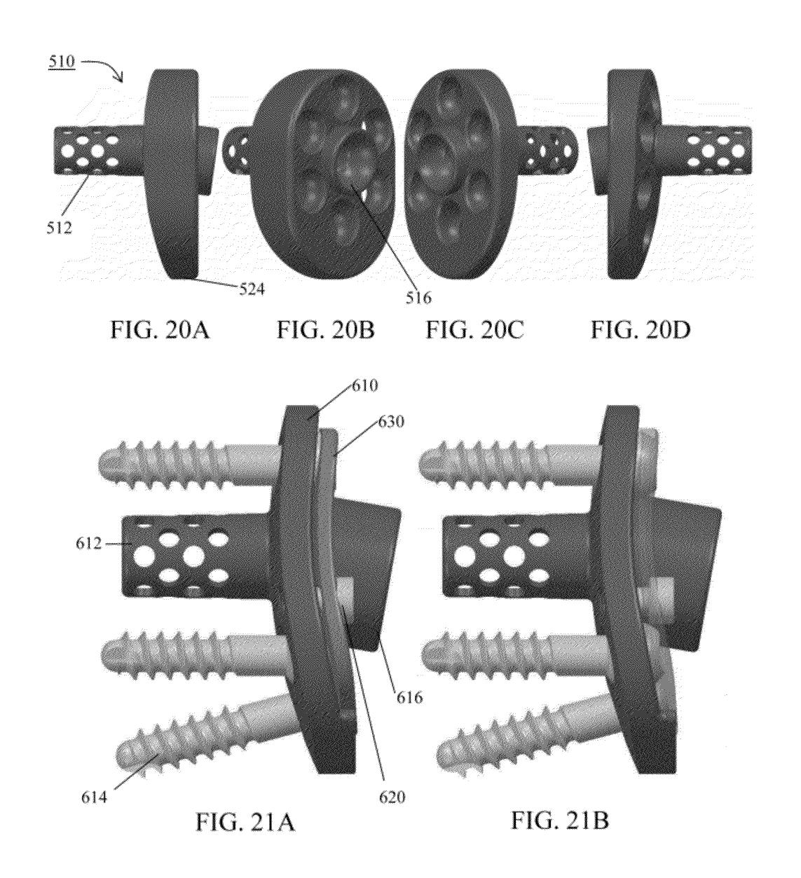

FIGS. 20A-20D present an embodiment of a glenoid plate 510 in which the posterior portion of the glenoid plate 510 is augmented by 10°. In an embodiment, the glenoid plate 510 is substantially oval in shape. In an embodiment, the glenoid plate 510 is substantially circular in shape. The glenoid plate 510 fixes directly to the glenoid bone (or malformed/eroded glenoid bone) with locking/compression screws and a pressfit male boss. The glenoid plate 510 can be shaped, angled, or augmented (e.g. posteriorly, superiorly, etc) in such a way to optimize fixation for each patient's glenoid morphology and help to restore native glenoid version and native glenoid inclination intraoperatively should a surgeon desire. The glenoid plate 510 includes a spherical tapered surface 516 that mates with a locking spherical articulation of an adjustment plate of the present invention. Glenoid plate 510 includes a central stem 512. The stem 512 is a superiorly shifted fixation structure. In order to conserve the much needed glenoid bone, the central stem 512 is shifted superiorly (e.g., by 4 mm)—enabling a surgeon to maintain the traditional surgical technique with the reverse as would be performed for total shoulder arthroplasty (i.e. drilling a hole in the center of the glenoid bone where the defect would occur; thereby, conserving bone). In an embodiment, the central stem 512 is sufficiently superiorly shifted from the central point of the vertical dimension so that, when the glenoid plate 510 is implanted in a glenoid bone so that the central stem 512 is positioned in a center of the glenoid bone, a distal rim 524 of the glenoid plate 510 is aligned with a distal edge of an articular surface of the glenoid bone. In an embodiment, the glenoid plate hole positions are designed to allow conversion of a traditional peg and keel glenoid. In some embodiments, in order to improve glenoid fixation, the central stem 512 allows bone “through-growth” using inductive/conductive bone graft. Bone graft can be placed into the stem prior to securing the plate with screws and/or after (e.g., by injecting the graft through a syringe in the top of the plate). The bone through-growth fixation stem 512 can be either cylindrical (e.g., to revise a peg glenoid) or non-cylindrical (e.g., to revise a keel glenoid). In some embodiments, the glenoid plate 510 may incorporate several other features which should work to conserve glenoid bone and/or improve fixation. The glenoid plate 510 may have a curved-back to minimize the amount of bone removed for implantation, (compared to the flat-back glenoid plate designs, as the native glenoid bone is also curved). Additionally, one or more screw holes in the glenoid plate 510 may have a female spherical feature which mates with the male spherical head of the compression screw. Doing so may allow for each compression screw to be angled/oriented in any desired direction—thereby improving the possibility of screw purchase. Additionally, one or more of the screw holes in the glenoid plate 510 may have a threaded feature for attachment of a locking cap. The cap screw may have a female spherical feature which compresses the spherical head of the compression screw; thereby locking it to the plate at whatever angle/orientation the screw was inserted into the bone (preventing it from backing out).

FIGS. 21 and 21B show components of an embodiment of a glenoid assembly (a glenosphere and an adjustment plate are not illustrated) for use with a reverse shoulder prosthesis of the present disclosure. As illustrated in FIGS. 21A and 21B, a locking plate 630 is secured to a glenoid plate 610 with 2 small set screws 620 instead of being threaded with one locking ring. The locking plate 630 is used to secure polyaxial compression screws 614 (which, in an embodiment, can be inserted into the glenoid/scapula at approximately 8°) to the glenoid plate 610 and to prevent the polyaxial compression screws 614 from backing out (e.g. losing their compression). The locking plate 630 secures/locks all the polyaxial compression screws 614 at the same time. The glenoid plate 610 fixes directly to the glenoid bone (or malformed/eroded glenoid bone) with locking/compression screws and a pressfit male boss. The glenoid plate 610 can be shaped, angled, or augmented (e.g. posteriorly, superiorly, etc) in such a way to optimize fixation for each patient's glenoid morphology and help to restore native glenoid version and native glenoid inclination intraoperatively should a surgeon desire. The glenoid plate 610 includes a spherical tapered surface 616 that mates with a locking spherical articulation of an adjustment plate of the present invention. The glenoid plate 610 includes a body portion having a front and a back, and a central stem 612 that is a superiorly shifted fixed structure. In order to conserve the much needed glenoid bone, the glenoid plate 610 according to an embodiment is designed so that the central stem 612 is shifted superiorly (e.g., by 4 mm)—enabling a surgeon to maintain the traditional surgical technique with the reverse as would be performed for total shoulder arthroplasty (i.e. drilling a hole in the center of the glenoid bone where the defect would occur; thereby, conserving bone). The body portion of the glenoid plate 610 has a vertical dimension, wherein a central point of the vertical dimension divides the body portion into an upper half and a lower half, wherein the central stem 612 has a central longitudinal axis, and wherein the central stem 612 extends from the body portion from a position on the body portion such that the central longitudinal axis of the central stem 612 is sufficiently superiorly shifted from the central point of the vertical dimension so that when the glenoid plate 610 is implanted in a glenoid bone so that the central stem 612 is positioned in a center of the glenoid bone, a distal rim of the glenoid plate 610 is aligned with a distal edge of an articular surface of the glenoid bone. In an embodiment, glenoid plate hole positions are designed to allow conversion of a traditional peg and keel glenoid. In some embodiments, in order to improve glenoid fixation, the central stem 612 allows bone “through-growth” using inductive/conductive bone graft. Bone graft can be placed into the stem prior to securing the plate with screws and/or after (e.g., by injecting the graft through a syringe in the top of the plate). The bone through-growth fixation stem 612 can be either cylindrical (e.g., to revise a peg glenoid) or non-cylindrical (e.g., to revise a keel glenoid). In some embodiments, the glenoid plate 610 of the present disclosure may incorporate several other features which should work to conserve glenoid bone and/or improve fixation. The glenoid plate 610 may have a curved-back to minimize the amount of bone removed for implantation, (compared to the flat-back glenoid plate designs, as the native glenoid bone is also curved). Additionally, one or more screw holes in the glenoid plate may have a female spherical feature which mates with the male spherical head of the compression screw. Doing so may allow for each compression screw to be angled/oriented in any desired direction—thereby improving the possibility of screw purchase.

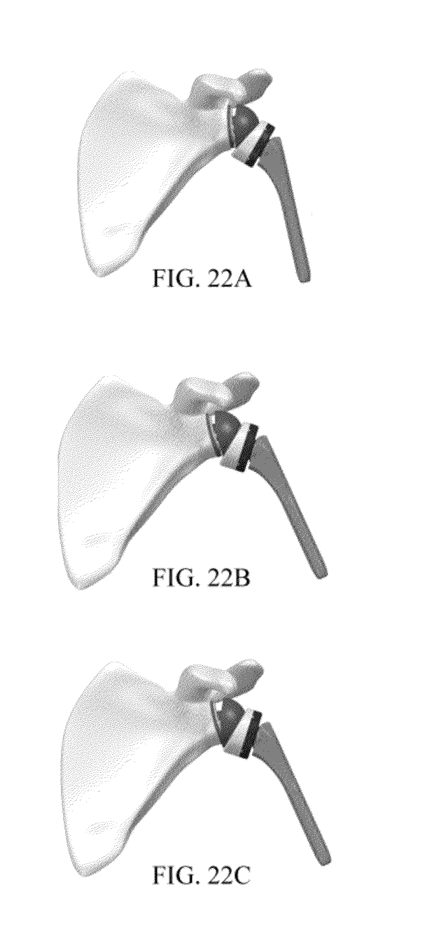

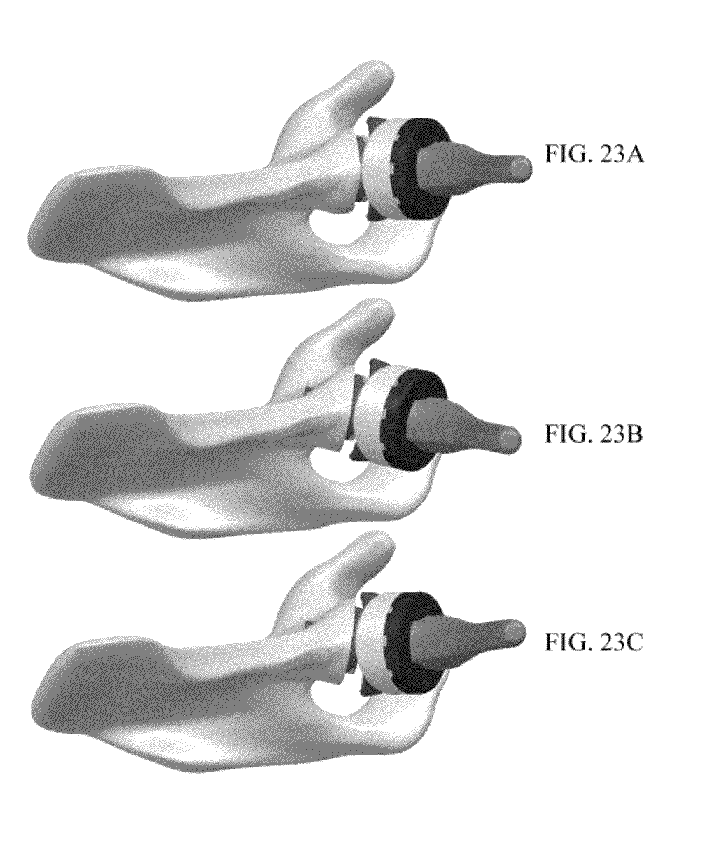

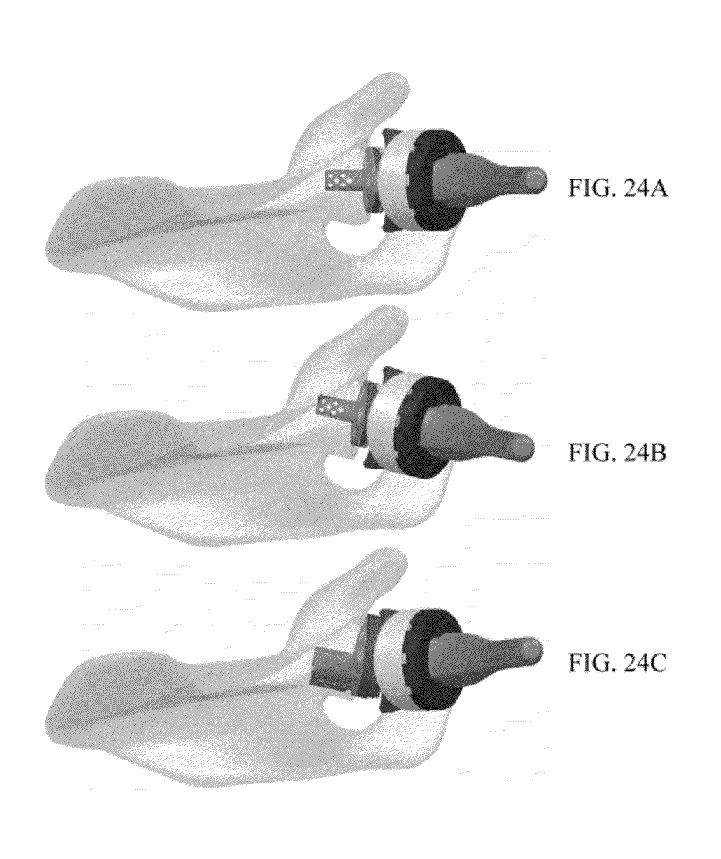

To better illustrate the benefit of these described adjustable reverse shoulder prostheses, the adjustable reverse shoulder prostheses were assembled to a computer model of the scapula with superior glenoid wear (FIGS. 22A-22C) and with posterior glenoid wear (FIGS. 23A-23C and FIGS. 24A-24C). FIGS. 23A-23C show schematic illustrations of the use of an embodiment of a reverse shoulder prosthesis of the present disclosure having an adjustable glenoid assembly to correct glenoid inclination for a superiorly worn glenoid (top image depicts adjustable glenoid plate when oriented in neutral position on a normal glenoid; middle image depicts adjustable glenoid plate when oriented in neutral position on a superiorly worn glenoid (not superior tilt of glenosphere due to superiorly worn bone condition); bottom image depicts adjustable glenoid plate when oriented inferiorly by 12 degrees on a superiorly worn glenoid). FIGS. 23A-23C demonstrate how inferiorly orienting the glenosphere on a superiorly worn glenoid can correct glenoid inclination, reduce humeral impingement on the glenoid, and reposition the humeral component to a more anatomic position.

FIGS. 23A-23C show schematic illustrations of the use of an embodiment of a reverse shoulder prosthesis of the present disclosure having an adjustable glenoid assembly to correct glenoid version for a posteriorly worn glenoid. (top image depicts adjustable glenoid when oriented in neutral position on a normal glenoid; middle image depicts adjustable glenoid when oriented in neutral position on a posteriorly worn glenoid; bottom image depicts adjustable glenoid when oriented anteriorly by 12 degrees on a posteriorly worn glenoid.) Note that the anteriorly oriented component corrects the positioning of the humeral component so that it is oriented with the long axis of the scapula (as in the top image). FIGS. 23A-23C demonstrate how anteriorly positioning the glenosphere on a posteriorly worn glenoid can correct the version/positioning of the humeral prosthesis.

FIGS. 24A-24C show schematic illustrations of the use of an embodiment of a reverse shoulder prosthesis of the present disclosure having an adjustable glenoid assembly to position the cage pegs to a location that does not perforate the anterior glenoid (as seen in FIG. 3) in a posteriorly worn glenoid (top image depicts adjustable glenoid when oriented in neutral position on a normal glenoid; middle image depicts adjustable glenoid when oriented in neutral position on a posteriorly worn glenoid; bottom image depicts adjustable glenoid when oriented anteriorly by 12 degrees on a posteriorly worn glenoid and with the modular central peg moved to the posterior position of the glenoid plate so that the anterior scapula is not perforated.) Note that in the bottom image the posteriorly positioned cage pegs do not perforate the anterior scapula in the posteriorly worn glenoid. FIGS. 24A-24C demonstrate how modularly connecting the glenoid fixation surfaces to a location other than the center of the glenoid (e.g. to a posterior position) can prevent their anterior perforation on a posteriorly worn glenoid. Note that the positioning of these modular fixation surfaces can also be used to facilitate grafting.

A reverse total shoulder arthroplasty method of the present disclosure includes fixating a glenoid plate to a glenoid bone; locking an adjustment plate, configured for interfacing with both the glenoid plate and a glenosphere, to the glenoid plate, wherein the adjustment plate is configured for angular orientation or positional change relative to the glenoid plate; connecting a glenosphere to the adjustment plate; and independently adjusting an angular orientation and position of the glenosphere relative to the fixated glenoid plate. In an embodiment, the glenoid plate includes a body portion and a stem portion, wherein the body portion has a central horizontal axis and a central vertical axis, wherein the central horizontal axis and the central vertical axis intersect at a central point that divides the body portion into an upper half, a lower half, a right half and a left half, wherein the stem portion has a central longitudinal axis perpendicular to the central vertical axis of the body portion, and wherein the stem portion extends from a back face of the body portion from a position on the body portion such that the central longitudinal axis of the stem portion is superiorly shifted from the central point of the body portion along the central vertical axis. In an embodiment, when the glenoid plate is implanted in the glenoid bone, the stem portion is positioned in a center of the glenoid bone, and a distal rim of the glenoid plate is aligned with a distal edge of an articular surface of the glenoid bone. In an embodiment, the stem portion of the glenoid plate affixes the glenoid plate to the glenoid bone. In an embodiment, additional modular fixation structures are intraoperatively positioned through holes of the glenoid plate into user determined locations of the glenoid bone. In an embodiment, one or more compression screws help fixate the glenoid plate to the glenoid bone, and a locking ring is used to simultaneously lock each of the compression screws.

A reverse total shoulder arthroplasty method of the present disclosure includes providing a glenoid plate having a body portion with a plurality of through holes; positioning a back face of the glenoid plate on an articular surface of a glenoid bone; and securing the glenoid plate to the glenoid bone by positioning at least one bone through-growth cage through one of the through holes and into the glenoid bone. In an embodiment, the method further include locking an adjustment plate, configured for interfacing with both the glenoid plate and a glenosphere, to the glenoid plate, wherein the adjustment plate is configured for angular orientation or positional change relative to the glenoid plate. In an embodiment, the glenoid plate includes a stem portion extending from the back face of the body portion, wherein the body portion has a central horizontal axis and a central vertical axis, wherein the central horizontal axis and the central vertical axis intersect at a central point that divides the body portion into an upper half, a lower half, a right half and a left half, wherein the stem portion has a central longitudinal axis perpendicular to the central vertical axis of the body portion, and wherein the stem portion extends from a back face of the body portion from a position on the body portion such that the central longitudinal axis of the stem portion is superiorly shifted from the central point of the body portion along the central vertical axis. In an embodiment, when the glenoid plate is implanted in the glenoid bone, the stem portion is positioned in a center of the glenoid bone, and a distal rim of the glenoid plate is aligned with a distal edge of an articular surface of the glenoid bone. In an embodiment, additional modular fixation structures are intraoperatively positioned through the through holes of the glenoid plate into user determined locations of the glenoid bone. In an embodiment, one or more compression screws help fixate the glenoid plate to the glenoid bone, and a locking ring is used to simultaneously lock each of the compression screws.

A method of the present disclosure for implanting an adjustable glenoid assembly of a reverse shoulder prosthesis in a glenoid bone includes reaming a surface of the glenoid bone perpendicular to an eroded surface of the glenoid bone so as to conserve maximum amount of remaining glenoid bone; drilling a hole in the glenoid bone, the hole being sufficiently sized for accepting one or more fixation structures of a glenoid plate; impacting the glenoid plate into the hole; drilling at least one pilot hole in the glenoid bone, the hole being sufficiently sized for accepting one or more compression screws; threading the one or more compression screws through the glenoid plate along an axis of each of the pilot holes into the glenoid bone; threading a locking ring to the glenoid plate to lock each of the compression screws, wherein each of the compression screws are simultaneously locked; connecting an adjustment plate, having an articulation and a connection, to the glenoid plate so as to permit at least one of angular orientation or positional change; orienting and positioning a glenosphere; tightening the a connecting the adjustment plate to the glenoid plate; and impacting the glenosphere onto the connection of the adjustment plate. In an embodiment, the glenosphere is oriented and positioned to a desired position that maximizes stability. In an embodiment, the center of rotation, version, inclination, or both angles of the glenosphere are adjusted intraoperatively. In an embodiment, the method further includes an initial step of assessing the type and magnitude of glenoid wear prior to reaming a surface of the glenoid bone. In an embodiment, the fixation structure of the glenoid plate is fixed to the glenoid plate. In an embodiment, a fixation structure of the glenoid plate is modular and positioned within any one or more of the holes in the glenoid plate at multiple different locations on the glenoid. In an embodiment, the spherical articulation of the adjustment plate is sufficiently designed to allow for an about 12 degrees of angular orientation. In an embodiment, the spherical articulation of the adjustment plate is sufficiently designed to allow for a several millimeter change in position in the center of rotation of the glenoid assembly either by means of the offset taper of the adjustment plate or by means of differing center of rotation positions between the adjustment plate spherical taper and the glenosphere. In an embodiment, maximizing stability results in at least one of balancing of soft tissue, eliminating impingement (e.g. scapular notching), or maximizing range of motion.

While a number of embodiments of the present disclosure have been described, it is understood that these embodiments are illustrative only, and not restrictive, and that many modifications may become apparent to those of ordinary skill in the art. For example, any element described herein may be provided in any desired size (e.g., any element described herein may be provided in any desired custom size or any element described herein may be provided in any desired size selected from a “family” of sizes, such as small, medium, large). Further, one or more of the components may be made from any of the following materials: (a) any biocompatible material (which biocompatible material may be treated to permit surface bone ingrowth or prohibit surface bone ingrowth—depending upon the desire of the surgeon); (b) a plastic; (c) a fiber; (d) a polymer; (e) a metal (a pure metal such as titanium and/or an alloy such as Ti—Al—Nb, Ti-6Al-4V, stainless steel); (f) any combination thereof. Further still, the metal construct may be a machined metal construct. Further still, various cage designs (e.g. square/elliptical/angled cages) may be utilized. Further still, various keel designs (e.g. anterior/posterior keel, medial/lateral keel, dorsal fin keel, angled keel) may be utilized. Further still, the prosthesis may utilize one or more modular elements. Further still, any desired number of cages(s) and/or keel(s) may be utilized with a given prosthesis. Further still, any number of male features that could dig into the bone so that initial/supplemental fixation can be improved may be utilized with a given prosthesis. Further still, any number of bone screws (e.g., such as for initial fixation and/or such as for supplemental fixation) may be utilized with a given prosthesis. Further still, any steps described herein may be carried out in any desired order (and any additional steps may be added as desired and/or any steps may be deleted as desired).

All patents, patent applications, and published references cited herein are hereby incorporated by reference in their entirety. It will be appreciated that several of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

What is claimed is:

1. A glenoid assembly comprising:
a glenoid plate configured for fixation to a glenoid bone for a reverse shoulder prosthesis;
a glenosphere configured for connection to the glenoid plate; and
an adjustment plate,
wherein the adjustment plate has a connection for directly engaging the glenosphere, and
wherein the adjustment plate has an articulation for directly engaging the glenoid plate at a variable angular orientation.

2. The glenoid assembly of claim 1 wherein the adjustment plate interfaces with the glenosphere and the glenoid plate to indirectly connect the glenosphere to the glenoid plate.

3. The glenoid assembly of claim 1 wherein the glenoid plate includes a body portion and a stem portion,
wherein the body portion of the glenoid plate has a front face, a back face and a thickness therebetween,
wherein the body portion of the glenoid plate has a central horizontal axis and a central vertical axis,
wherein the central horizontal axis and the central vertical axis intersect at a central point that divides the body portion into an upper half, a lower half, a right half and a left half,
wherein the stem portion has a central longitudinal axis perpendicular to the central vertical axis of the body portion, and
wherein the stem portion extends from the back face of the body portion from a position on the body portion such that the central longitudinal axis of the stem portion is superiorly shifted from the central point of the body portion along the central vertical axis.

4. The glenoid assembly of claim 3 wherein the stem portion allows bone through-growth using inductive/conductive bone graft.

5. The glenoid assembly of claim 1 wherein an articular surface of the glenosphere is configured for mating with a humeral liner of an adjustable reverse total shoulder prosthesis.

6. The glenoid assembly of claim 1 wherein the connection of the adjustment plate is tapered.

7. The glenoid assembly of claim 1 wherein the connection of the adjustment plate is offset so as to permit the glenosphere to be positioned in an offset location of about 3 mm relative to the glenoid plate.

8. The glenoid assembly of claim 1 wherein the articulation of the adjustment plate is spherical.

9. The glenoid assembly of claim 1 further comprising a locking ring or plate configured for securing polyaxial compression screws to the glenoid plate and for preventing the polyaxial compression screws from backing out of the glenoid plate.

10. The glenoid assembly of claim 1 wherein the glenosphere has an anterior side and a posterior side that is chamfered.

11. The glenoid assembly of claim 1 wherein the glenoid plate includes a tapered surface that mates and locks with the articulation of the adjustment plate for directly engaging the glenoid plate at a variable angular orientation.

12. The glenoid assembly of claim 3 wherein the body portion of the glenoid plate includes a plurality of through holes for inserting one or more modular fixation structures selected from one of polyaxial compression screws, bone through-growth cages, or combinations thereof.

13. A glenoid assembly comprising:
a glenoid plate configured for fixation to a glenoid bone for a reverse shoulder prosthesis,
wherein the glenoid plate includes a body portion, and
wherein the body portion includes a plurality of through holes;
at least one bone through-growth cage positioned through one of the through holes; and
a glenosphere configured for connection to the glenoid plate.

14. The glenoid assembly of claim 13 further comprising an adjustment plate,
wherein the adjustment plate has a connection for directly engaging the glenosphere, and
wherein the adjustment plate has an articulation for directly engaging the glenoid plate at a variable angular orientation.

15. The glenoid assembly of claim 13 wherein the adjustment plate interfaces with the glenosphere and the glenoid plate to indirectly connect the glenosphere to the glenoid plate.

16. The glenoid assembly of claim 13 wherein the glenoid plate further includes a stem portion extending from a back face of the body portion, and wherein the stem portion is configured to be disposed within a space formed in the glenoid bone of a patient.

17. The glenoid assembly of claim 13 further comprising at least one polyaxial compression screw positioned through one of the through holes.

18. The glenoid assembly of claim 17 further comprising a locking ring or plate configured for securing the at least one polyaxial compression screw to the glenoid plate and for preventing the polyaxial compression screw from backing out of the glenoid plate.

19. The glenoid assembly of claim 13 wherein the glenoid plate includes a tapered surface that mates and locks with the articulation of the adjustment plate for directly engaging the glenoid plate at a variable angular orientation.

20. The glenoid assembly of claim 13 wherein an articular surface of the glenosphere is configured for mating with a humeral liner of an adjustable reverse shoulder prosthesis.
